(19) United States
(12) Patent Application Publication
(10) Pub. No.: US 2023/0255640 A1
(43) Pub. Date: Aug. 17, 2023

(54) INFLATABLE RADIAL ARTERY COMPRESSION DEVICE WITH REINFORCED BACKER PLATE

(71) Applicant: Merit Medical Systems, Inc., South Jordan, UT (US)

(72) Inventors: Jacquelyn Monroe, Highland, UT (US); Aaron Hopkinson, Herriman, UT (US)

(21) Appl. No.: 18/166,721

(22) Filed: Feb. 9, 2023

Related U.S. Application Data
(60) Provisional application No. 63/267,864, filed on Feb. 11, 2022.

(51) Int. Cl. A61B 17/132

(52) U.S. Cl. CPC A61B 17/1325

(57) ABSTRACT

Devices and methods used to provide hemostasis at a radial artery access site are disclosed. The devices include a backer plate, an inflatable chamber, and a wristband. The backer plate includes a reinforced section and flex points. In a flexed state the backer plate is flexed at the flex points and the reinforced section is planar to provide a planar compressive surface over the radial artery access site.

### RELATED APPLICATIONS

This application claims priority to U.S. Provisional Application No. 63/267,864, filed on Feb. 11, 2022 and titled, “INFLATABLE RADIAL ARTERY COMPRESSION DEVICE WITH REINFORCED BACKER PLATE,” which is hereby incorporated by reference in its entirety.

### TECHNICAL FIELD

The present disclosure relates generally to the field of medical devices. More particularly, some embodiments relate to compression devices, including radial artery compression devices with an inflatable chamber and a reinforced backer plate.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a radial artery compression device secured to a patient's wrist.

FIG. 2 is a perspective view of the radial artery compression device of FIG. 1.

FIG. 3A is a top perspective view of an embodiment of a backer plate of the radial artery compression device of FIG. 1.

FIG. 3B is a bottom perspective view of the backer plate of FIG. 3A.

FIG. 3C is a cross-sectional view of another embodiment of a backer plate.

FIG. 4 is a perspective of an embodiment of an inflatable chamber of the radial artery compression device of FIG. 1.

FIG. 5A is a bottom perspective view of an embodiment of an assembly of the backer plate and inflatable chamber of FIG. 1 in a non-flexed state.

FIG. 5B is a cross-sectional view of the assembly of FIG. 5A in a non-flexed state.

FIG. 5C is a cross-sectional view of the assembly of FIG. 5A in a flexed state.

FIG. 6A is an embodiment of a location indicium of the radial artery compression device of FIG. 1.

FIG. 6B is another embodiment of a location indicium of the radial artery compression device of FIG. 1.

FIG. 7A is a cross-sectional view of the radial artery compression device of FIG. 1 partially disposed on a patient wrist over a radial artery access site.

FIG. 7B is a cross-sectional view of the radial artery compression device of FIG. 1 secured to the patient wrist over the radial artery access site.

FIG. 7C is a cross-sectional view of the radial artery compression device of FIG. 1 inflated to apply a compressive force to the radial artery access site.

### DETAILED DESCRIPTION

Various medical procedures involve insertion of one or more elongate medical devices into the vasculature of a patient. Some such interventional procedures involve delivery of a medical device through a radial artery of the patient. Achieving hemostasis during and/or after an interventional procedure that involves puncturing the vasculature may be facilitated by compression. Certain embodiments within the scope of this disclosure relate to compression devices configured to compress the radial artery of a patient. Use of such devices, or analogous devices, to provide compression along other portions of the vasculature, including vasculature within the arm, leg, or other parts of the human body, are likewise within the scope of this disclosure. Accordingly, disclosure recited herein in connection with compression of the radial artery may be analogously applied to devices configured to compress other portions of the vasculature.

To facilitate hemostasis at the radial artery access site, pressure may be applied at an arteriotomy site that may be slightly upstream of a skin puncture site. Such pressure may prevent or reduce the leakage of blood from the arteriotomy site and promote hemostasis. Certain embodiments described herein facilitate the application of pressure to promote hemostasis at a radial artery access site.

Embodiments herein describe radial artery compression devices and methods to assist in providing hemostasis of a radial artery access site following an intravascular interventional procedure. The device can be secured to either a patient's left wrist or a patient's right wrist. In some embodiments within the scope of this disclosure, the devices include a backer plate or frame, an inflatable chamber, and a wristband. The backer plate includes a middle portion having a reinforced section. End portions of the backer plate are angled downwardly and outwardly relative to the middle portion. The backer plate is configured to flex or bend at flex points adjacent the reinforced section when a downward force is applied to the end portions by the wristband when the radial artery compression device is secured to the patient's wrist. Flexing of the backer plate allows the backer plate to conform to the contour of the patient's wrist and provide comfort to the patient. When the backer plate is flexed, the reinforced section remains substantially planar to provide a rigid support for the inflatable chamber when inflated to apply compression to the radial artery access site. One end of the backer plate includes a cinch bar configured to couple with the wristband when the radial artery compression device is secured to the patient's wrist. In some embodiments, the wristband includes a hook-and-loop material to provide for adjustability to accommodate patients' wrists of a variety of circumferences.

In use, in embodiments within the scope of this disclosure, the radial artery compression device is secured to the patient's wrist with the backer plate and inflatable chamber disposed over the radial artery access site, the wristband wrapped around the patient's wrist and the cinch bar, and a free end of the wristband coupled to a body of the wristband. Downward forces are applied to the ends of the backer plate causing the backer plate to flex at the flex points while the reinforced section remains substantially planar. A fluid (e.g., air or saline) is injected into the inflatable chamber to inflate or pressurize the inflatable chamber resulting in compression of the radial artery access site to achieve hemostasis. In certain embodiments, the inflatable chamber may be deflated or de-pressurized incrementally over a period of time.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

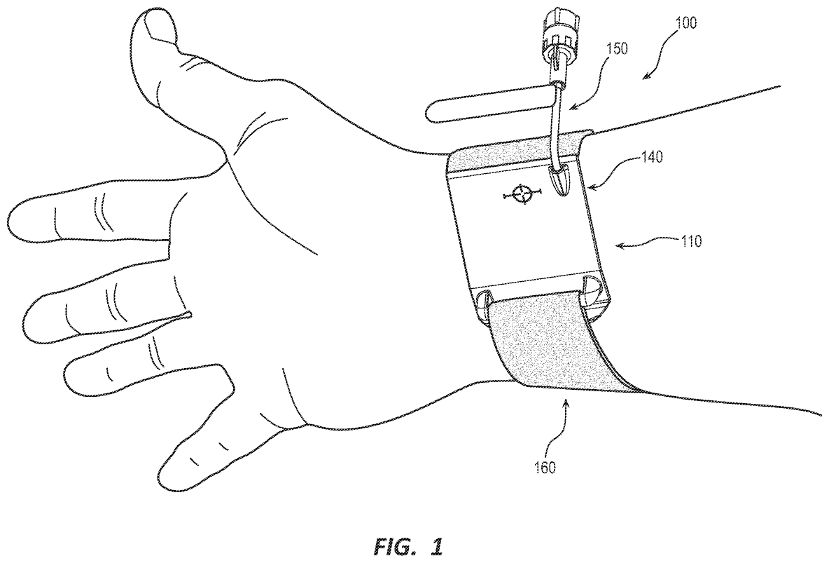

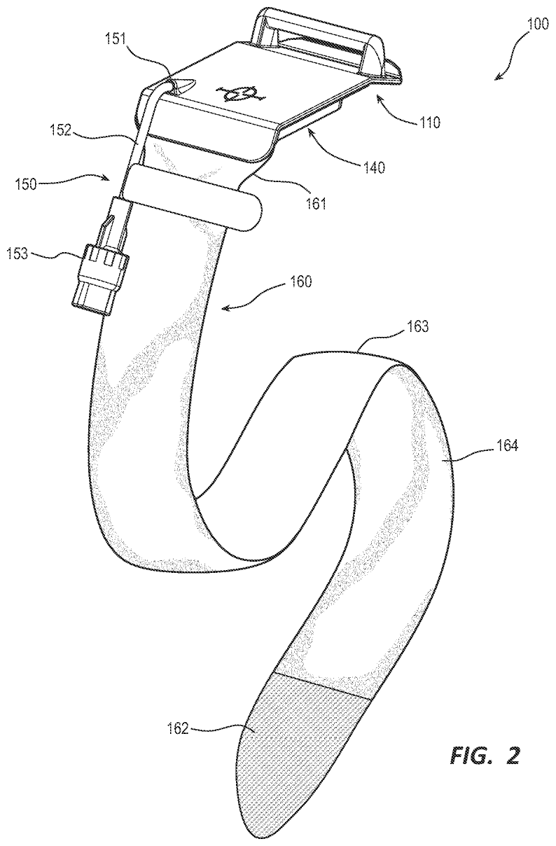

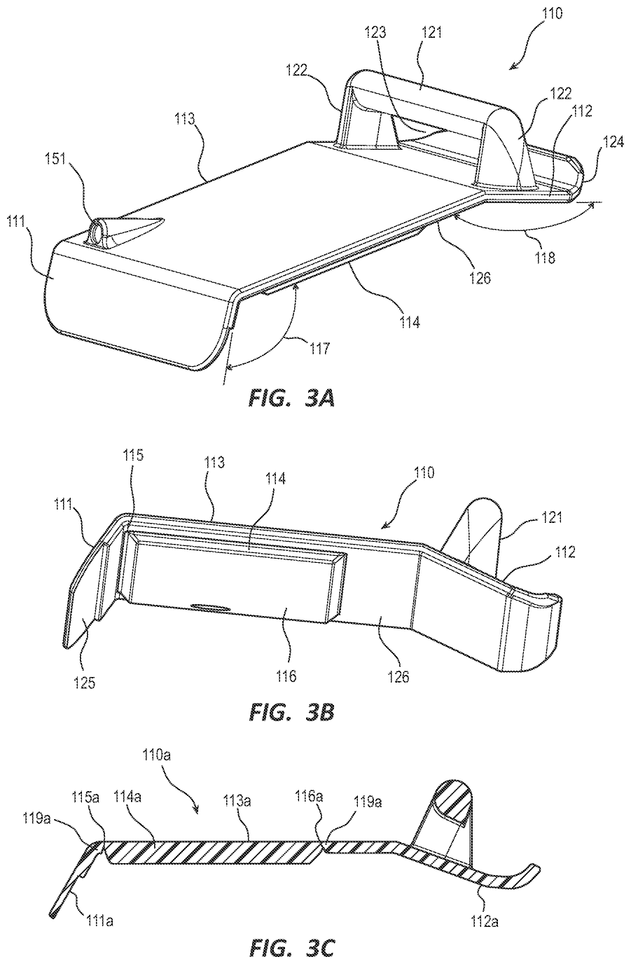

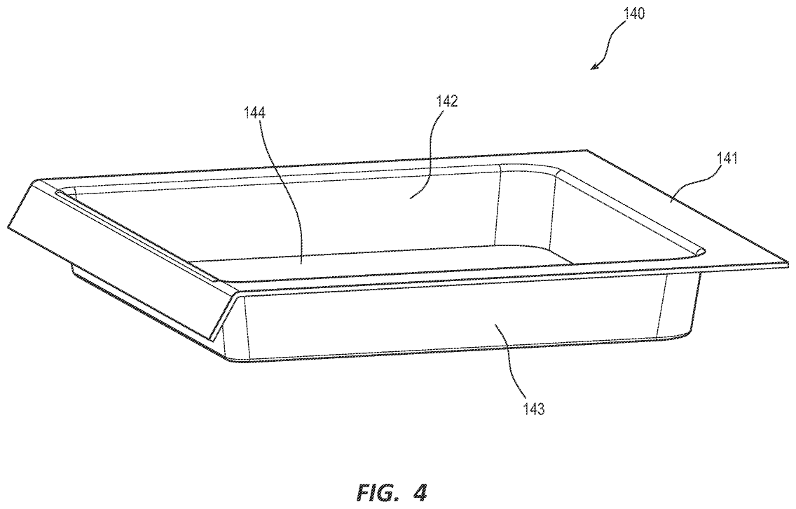

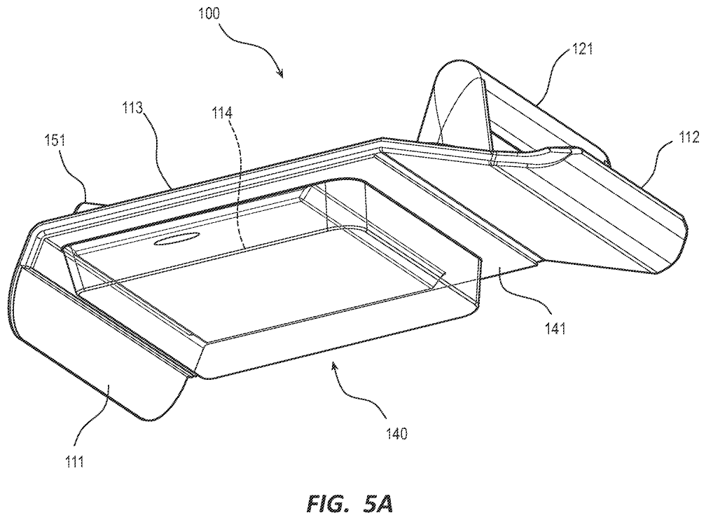

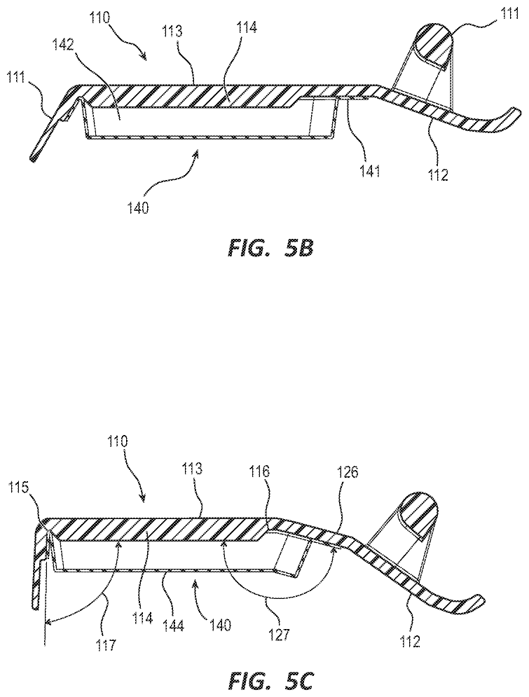

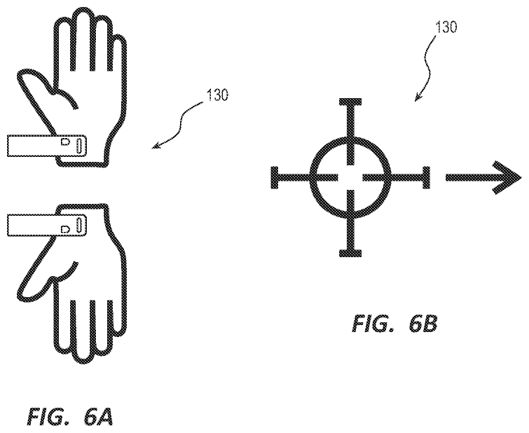

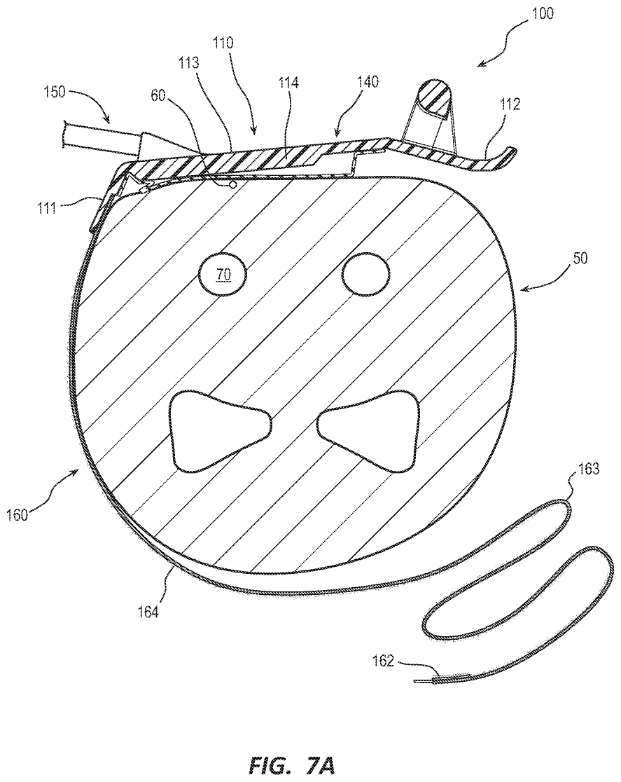

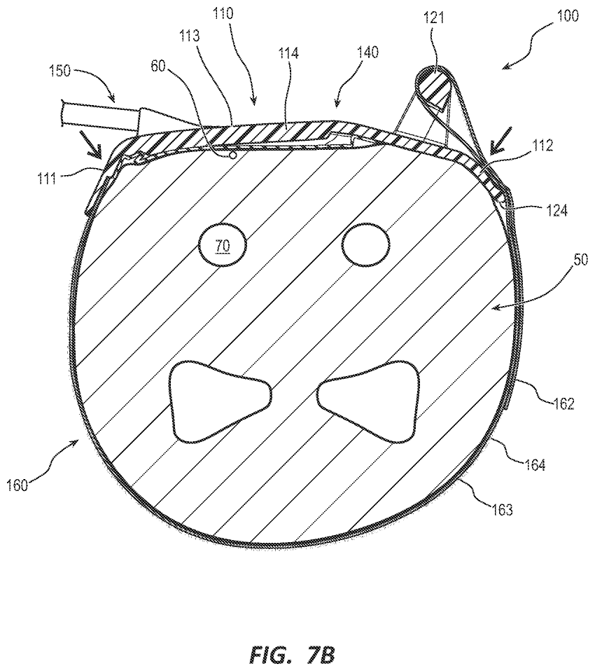

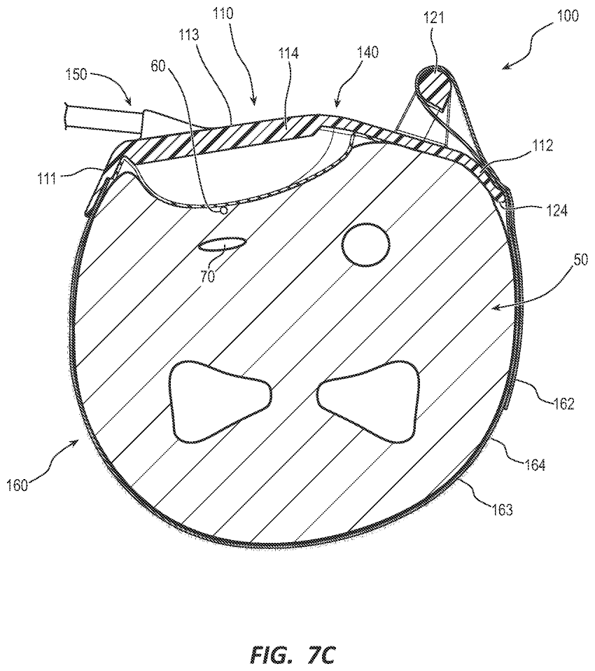

FIGS. 1 and 2 illustrate an embodiment of a radial artery compression device. FIGS. 3A and 3B illustrate an embodiment of a backer plate or frame of the radial artery compression device. FIG. 3C illustrates another embodiment of a backer plate of the radial artery compression device. FIG. 4 illustrates an embodiment of an inflatable chamber of the radial artery compression device. FIGS. 5A and 5B illustrate an embodiment of an assembly of the backer plate and the inflatable chamber in a non-flexed state. FIG. 5C illustrates the assembly of the backer plate and the inflatable chamber in a flexed state. FIGS. 6A and 6B illustrate embodiments of location or orientation indicia of the radial artery compression device. FIGS. 7A-7C illustrate a method of use of the radial artery compression device for achieving hemostasis at a radial artery access site. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

FIG. 1 illustrates an embodiment of a radial artery compression device 100 secured to a patient's wrist (i.e., right wrist) and disposed over a radial artery access site to provide compression to the access site to achieve hemostasis of an arteriotomy of the radial artery following a diagnostic or therapeutic intravascular interventional procedure. The diagnostic procedure may include coronary angiogram or similar type of procedure and the therapeutic procedure may include a percutaneous coronary intervention including a balloon angioplasty and stent placement. Other procedures are contemplated within the scope of this disclosure. The illustrated embodiment of the radial artery compression device 100 includes a backer plate or frame 110, an inflatable chamber 140, an inflation port 150, and a wristband 160.

As illustrated in FIG. 2, the inflation port 150 is coupled to the backer plate 110 in fluid communication with the inflatable chamber 140. The inflation port 150 can be utilized to inflate or pressurize and deflate or de-pressurize the inflatable chamber 140 by facilitation of injection and withdrawal of a fluid (e.g., air or saline) into the inflatable chamber 140 using a fluid delivery device, such as a syringe. The inflation port 150 includes a passage 151 through the backer plate 110, a tubing 152 coupled to the passage 151 at a first end, and a fluid control valve 153 coupled to the tubing 152 at a second end. The tubing 152 can be flexible to allow for manipulation of the inflation port 150 during use. The fluid control valve 153 is configured to selectively open and close to allow fluid to flow through the fluid control valve 153 into or from the inflatable chamber 140 when in an open state and to retain fluid within the inflatable chamber 140 when in a closed state. In the depicted embodiment, the fluid control valve 153 is a Luer actuated valve including a valve member actuatable by a male Luer fitting. In other embodiments, the fluid control valve 153 may be any suitable type of valve configured to be selectively transitioned from a closed state to an open state either manually or passively. For example, the fluid control valve 153 may be a two-way stopcock, a pinch clamp, a roller clamp, etc.

As further illustrated in FIG. 2, the wristband 160 includes a first end 161 fixedly coupled to the backer plate 110, a second or free end 162 disposed opposite the first end 161, and a body 163 extending between the ends 161, 162. The wristband 160 may include a selective coupling member 164 configured to facilitate selective securement of the radial artery compression device 100 to the patient. The selective coupling member 164 can be adjustable to accommodate patient wrists of differing circumference. In the depicted embodiment, the selective coupling member 164 includes a hook-and-loop material wherein the loop portion of the material is disposed on an outer surface of the body 163 and the hook portion of the material is disposed on an inner surface of the free end 162. In this configuration, the body 163 can be wrapped around the patient's wrist, coupled to the backer plate 110, and the free end 162 folded back on the body 163 to selectively couple the free end 162 to the body 163. In other embodiments, the selective coupling member 164 may include a buckle, a snap, a button, a clamp, etc.

FIGS. 3A and 3B illustrate an embodiment of the backer plate 110. As illustrated, the backer plate 110 is generally rectangular in shape having a first end portion 111, a second end portion 112, and a middle portion 113. The backer plate 110 may be formed from any suitable semi-rigid material. For example, the backer plate 110 may include, polyethylene, polypropylene, polyurethane, polyvinylchloride, nylon, ethylene-vinyl acetate, acrylonitrile butadiene styrene, etc. The first end portion 111 includes an inner surface 125 configured to fixedly engage the first end 161 of the wristband 160. The first end portion 111 is angled downwardly and outwardly at an angle 117 relative to the middle portion 113 to direct the wristband 160 downwardly around the patient's wrist. The angle 117 can range between about 105 degrees and about 125 degrees and can be about 120 degrees.

The second end portion 112 includes a cinch bar 121 supported by supports 122 and disposed transversely relative to a longitudinal axis of the backer plate 110. A slot 123 is defined between the cinch bar 121 and an upper surface of the second end portion 112. The slot 123 is configured to receive the free end 162 of the wristband 160 as the wristband 160 is wrapped around the cinch bar 121 to tighten and secure the radial artery compression device 100 to the patient's wrist. A lip 124 of the second end portion 112 may be angled or curved upwardly to prevent pinching of the patient's skin between the backer plate 110 and the wristband 160 when the radial artery compression device 100 is secured to the patient's wrist. The second end portion 112 is angled downwardly and outwardly at an angle 118 relative to the middle portion 113 to direct the wristband 160 downwardly around the patient's wrist. The angle 118 can range between about 145 degrees and about 165 degrees and can be about 160 degrees.

The middle portion 113 is substantially planar or non-curved and includes a reinforced section or viewing window 114 and a non-reinforced section 126. As depicted, the reinforced section 114 is disposed adjacent the first end portion 111 and the non-reinforced section 126 is disposed adjacent the second end portion 112. The reinforced section 114 includes a wall thickness that is greater than a wall thickness of the non-reinforced section 126 to increase a bending stiffness of the middle portion 113 in the area of the reinforced section 114. The bending stiffness of the reinforced section 114 may be between about 20% and about 200% greater than the bending stiffness of the non-reinforced section 126. The thickness of the reinforced section 114 may range from about 25% to about 100% greater than the wall thickness of the non-reinforced section 126. In another embodiment, the reinforced section 114 can include one or more longitudinally oriented ribs or structural supports extending from a lower surface of the middle portion 113 to increase the bending force of the reinforced section 114. Lateral edges of the reinforced section 114 may be angled as shown in FIGS. 3A and 3B. In other embodiments, the lateral edges can be perpendicular relative to a lower surface of the reinforced section 114. The port passage 151 may be disposed in the reinforced section 114.

A first flex or pivot or inflection point or joint 115 is disposed between the first end portion 111 and the reinforced section 114, and a second flex or pivot or inflection point or joint 116 is disposed between the reinforced section 114 and the non-reinforced section 126. The flex points 115, 116 are configured to allow the backer plate 110 to flex downward at the flex points 115, 116 while the reinforced section 114 remains substantially planar when downward forces are applied to the first and second end portions 111, 112 by the wristband 160. This configuration provides a compressive force to the radial artery access site by the reinforced section 114 while the remainder of the backer plate 110 substantially conforms to a contour of the patient's wrist.

FIG. 3C illustrates an alternate embodiment of a backer plate 110a. As illustrated, the backer plate 110a includes a first end portion 111a, a second end portion 112a, a middle portion 113a, and a reinforced section 114a, similar to the backer plate 110. The backer plate 110a includes weakened or thinned areas 119a disposed at first and second flex points 115a, 116a in a lower or upper surface of the backer plate 110a. The weakened areas 119a are configured to allow the backer plate 110a to flex at the flex points 115a, 116a at a relative low force applied to the first and second end portions 111a, 112a. In the illustrated embodiment, the weakened areas 119a include a V-groove disposed in the lower surface of backer plate 110a. When the backer plate 110a is flexed, the V-groove is configured to close.

FIG. 4 illustrates an embodiment of the inflatable chamber 140. As illustrated, the inflatable chamber 140 includes a sealing flange 141, a chamber 142, sidewalls 143, and a bottom wall 144. The depicted embodiment of the inflatable chamber 140 is rectangular in shape. In other embodiments, the inflatable chamber 140 may include any suitable shape, such as square, round, oval, elliptical, etc. The inflatable chamber 140 is formed by thermoforming a flexible material, such as a polyurethane, polyvinyl chloride, polyethylene terephthalate, etc. In another embodiment, the inflatable chamber 140 is formed by any suitable technique. For example, the inflatable chamber 140 may be formed by injection molding, stamping, etc. The chamber 142 is defined by the sidewalls 143 and the bottom wall 144 and may have a depth of between about one millimeter and about ten millimeters and may be about 5.1 millimeters. A resting volume of the chamber 142 may range from about two milliliters to about 12 milliliters and may be about five milliliters. The sealing flange 141 is coupled to the sidewalls 143 and extends circumferentially around the chamber 142. The sealing flange 141 is configured to be sealingly coupled to the backer plate 110 to prevent fluid from leaking from the chamber 142 when filled. A width of the sealing flange 141 may range between about 1.5 millimeters and about 13 millimeters and may be about 3.2 millimeters.

FIGS. 5A and 5B illustrate the inflatable chamber 140 coupled to the backer plate 110 in a non-flexed state. As illustrated, the inflation chamber 140 is located on a lower surface of the middle portion 113 between the first and second end portions 111, 112. The chamber 142 is disposed over the reinforced section 114 such that the reinforced section 114 is disposed within the chamber 142. The flange 141 is sealingly coupled to the lower surface of the middle portion 113 and the first end portion 111 using a radiofrequency (RF) welding technique. In other embodiments, the flange 141 may be sealingly coupled to the middle portion 113 and the first end portion 111 using any suitable technique, such as heat welding, sonic welding, adhesive bonding, solvent bonding, etc. The middle portion 113 is substantially planar in the non-flexed state.

FIG. 5C illustrates the inflation chamber 140 coupled to the backer plate 110 in a flexed state. As illustrated, the backer plate 110 is flexed downward at the first and second flex points 115, 116 wherein the middle portion 113 is non-planar and wherein the non-reinforced section 126 is pivoted or deflected or displaced downward relative to the reinforced section 114. A third angle 127 defined by the deflected non-reinforced section 126 and the reinforced section 114 can range between about zero degrees and about 20 degrees. The first end portion 111 may be displaced toward the middle portion 113 such that the angle 117 is decreased along a continuous range up to about 90 degrees or increased along a continuous range up to about 165 degrees. The bottom wall 144 of the inflation chamber 140 is substantially straight and free of wrinkles or creases when the backer plate 110 is in the flexed state. Additionally, when the backer plate 110 is in the flexed state over the radial artery access site, the backer plate 110 is configured to conform substantially to the contour of the patient's wrist such that the inflation chamber 140 applies a compression force directly over the radial artery access site to provide hemostasis and avoid a hematoma at the radial artery access site and to provide improved comfort to the patient.

FIGS. 6A and 6B illustrate embodiments of the indicium 130. The indicium 130 may be disposed on an upper surface of the backer plate 110 as illustrated in FIG. 2. The indicium 130 may be configured to indicate a desired orientation of the backer plate 110 when applied to the patient's wrist. For example, the indicium 130 of FIG. 6A indicates a direction of a wristband extending from a left hand and a right hand on a thumb side of the hands. The indicium 130 of FIG. 6B provides a circular target with targeting lines to be placed over the radial artery access site and an arrow indicating the direction of the wristband.

FIGS. 7A-7C illustrate the radial artery compression device 100 in use to apply a compressive force to a radial artery access site. As illustrated in FIG. 7A, the radial artery compression device 100 is disposed on an inner surface of a patient wrist 50 over a radial artery access site 60 and a radial artery 70. The radial artery compression device 100 may be disposed on either the patient left or right wrist with the wristband 160 extending from the backer plate 110 on the thumb side of the patient wrist 50. The reinforced section 114 and the inflatable chamber 140 are disposed over the radial artery access site 60 and the radial artery 70 with the backer plate 110 in the non-flexed state.

The wristband 160 is wrapped partially around the patient wrist 50. In some embodiments, the indicium (130 of FIG. 6A) may be utilized to properly orient the radial artery compression device 100 on the patient wrist 50 and over the radial artery access site 60 and the radial artery 70.

As illustrated in FIG. 7B, the wristband 160 is wrapped around the patient wrist 50, the free end 162 is passed through the slot 123, the wristband 160 is wrapped around the cinch bar 121, the wristband 160 is tightened around the patient wrist 50 to secure the radial artery compression device 100 to the patient wrist 50, and the free end 162 is coupled to the body 163 of the wristband 160. When the wristband 160 is tightened around the patient wrist 50, a downward force is applied to the first end portion 111 and the second end portion 112, as indicated by the arrows, resulting in flexing of the backer plate 110 at the first and second flex points 115, 116 to conform to a curved contour of the patient wrist 50. When the backer plate 110 flexes, the reinforced section 114 remains substantially planar.

As illustrated in FIG. 7C, the inflatable chamber 140 is inflated or pressurized by injecting a fluid (e.g., air or saline) through the inflation port 150 into the chamber 142. A volume of fluid ranging between about seven milliliters and about 12 milliliters can be injected into the chamber 142. When the inflatable chamber 140 is inflated, a compressive force is applied to the radial artery access site 60 and the radial artery 70 to achieve hemostasis or a cessation of blood flow at the radial artery access site 60. After a desired length of time the inflatable chamber 140 may be deflated or de-pressurized through the inflation port 150. In some embodiments, the inflatable chamber 140 may be deflated incrementally with a period of time between the increments. For example, two milliliters of fluid may be withdrawn from the inflatable chamber 140 every 15 minutes until the inflatable chamber 140 is deflated or emptied of the fluid.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method for achieving hemostasis at a radial artery access site may include one or more of the following steps: disposing a backer plate of a hemostasis device over a radial artery access site; positioning an inflatable chamber over the access site; wrapping a wristband around a circumference of a patient's wrist and coupling the wristband to the backer plate; tightening the wristband around the circumference of the patient's wrist; and flexing the backer plate at one or more of a first flex point and a second flex point. Other steps are also contemplated.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

“Fluid” is used in its broadest sense, to refer to any fluid, including both liquids and gases as well as solutions, compounds, suspensions, etc., which generally behave as fluids.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially planar” is recited with respect to a feature, it is understood that in further embodiments, the feature can have a precisely planar configuration.

The terms “a” and “an” can be described as one, but not limited to one. For example, although the disclosure may recite a housing having “a stopper,” the disclosure also contemplates that the housing can have two or more stoppers.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A radial artery compression device, comprising:
a backer plate comprising:
a first end portion;
a second end portion opposite the first end;
a middle portion disposed between the first end portion and the second end portion, the middle portion comprising a reinforced section,
wherein a first edge of the reinforced section defines a first flex point adjacent the first end portion and a second edge of the reinforced section defines a second flex point adjacent the second end portion, and
wherein the backer plate is configured to flex at the first and second flex points in a flexed configuration before flexing of the reinforced section when a downward force is applied to one or more of the first and the second end portions;
an inflatable chamber disposed on a lower surface of the backer plate; and
a wristband coupled to the backer plate and configured to secure the backer plate to a patient's wrist.

2. The device of claim 1, wherein a lower surface of the inflatable chamber is configured to be free of wrinkles when the backer plate is in the flexed configuration.

3. The device of claim 1, wherein the middle portion is straight when in a non-flexed configuration.

4. The device of claim 1, wherein the middle portion is non-curved when in a non-flexed state.

5. The device of claim 1, wherein the first end portion is angled relative to the middle portion at an angle ranging between 145 degrees and 165 degrees.

6. The device of claim 1, wherein the second end portion is angled relative to the middle portion at an angle ranging between 105 degrees and 125 degrees.

7. The device of claim 1, wherein a thickness of the reinforced section is greater than a thickness of one or more of the first end portion and the second end portion.

8. The device of claim 1, wherein the reinforced section comprises one or more longitudinally oriented ribs extending outward from the lower surface.

9. The device of claim 1, wherein a position of the reinforced section on the middle portion is biased toward the first end portion.

10. The device of claim 1,
wherein the first flex point comprises a first weakened portion disposed between the first end portion and the reinforced section, and
wherein the second flex point comprises a second weakened portion disposed between the second end portion and the reinforced section.

11. The device of claim 10, wherein one or more of the first and second weakened portions comprises a V-groove disposed within the inner surface.

12. The device of claim 1, wherein the inflatable chamber is coupled to the middle portion and disposed over the reinforced section.

13. The device of claim 1, wherein a volume of the inflatable chamber is five milliliters.

14. The device of claim 1, wherein the wristband is configured to apply the downward force to one or more of the first and second end portions when the radial artery compression device is secured to a patient's wrist.

15. The device of claim 1, wherein the backer plate comprises one or more indicia to indicate orientation of the backer plate relative to the radial artery when applied to the patient's wrist.

16. A method for achieving hemostasis at a radial artery access site, the method comprising:
disposing a backer plate of a hemostasis device over a radial artery access site;
positioning an inflatable chamber over the access site;
wrapping a wristband around a circumference of a patient's wrist and coupling the wristband to the backer plate;
tightening the wristband around the circumference of the patient's wrist; and
flexing the backer plate at one or more of a first flex point and a second flex point.

17. The method of claim 16, further comprising inflating the inflatable chamber, wherein a surface of the inflatable chamber is free of wrinkles when the backer plate is flexed and the inflatable chamber is inflated.

18. A frame for a radial artery compression device, comprising a middle portion comprising a reinforced section,
wherein a first edge of the reinforced section defines a first flex point and a second edge of the reinforced section defines a second flex point, and
wherein the frame is configured to flex at the first and second flex points before flexing of the reinforced section when a downward force is applied to the frame lateral to the first and second flex points.

19. The frame of claim 18, wherein the middle portion is straight when the frame is in a non-flexed configuration.

20. The frame of claim 18, wherein the middle portion is non-curved when the frame is in a non-flexed state.
